(19) United States
(12) Patent Application Publication
(10) Pub. No.: US 2001/0049554 A1
(43) Pub. Date: Dec. 6, 2001

(54) ENDOVASCULAR PROSTHESIS AND METHOD OF MAKING

(76) Inventors: CARLOS E. RUIZ, PASADENA, CA (US); ZVONIMIR KRAJCER, HOUSTON, TX (US); GERALD DORROS, SCOTTSDALE, AZ (US)

Correspondence Address: NICOLA PISANO, FISH AND NEAVE, 1251 AVENUE OF THE AMERICAS, NEW YORK, NY, 10020, US

(21) Appl. No.: 09/195,257

(22) Filed: Nov. 18, 1998

(51) Int. Cl. A61F 2/06

(52) U.S. Cl. 623/1.44; 623/1.35

(57) ABSTRACT

The present invention provides a prosthesis formed from a plurality of tubular layers members deployed in vivo using endovascular techniques and material. The layers define a lumen through a diseased portion of a vascular system. Each layer may be constructed using overlapping tubular members to provide a custom prosthesis. Subsequent prosthesis layers overlapping in the central portion of the lumen strengthen the prosthesis walls and may incorporate biocompatible materials having desirable properties.

### FIELD OF THE INVENTION

The field of the invention relates to prostheses for repairing occlusive and aneurysmal vascular disease, more particularly an in vivo constructed laminated endovascular system to repair occlusive and aneurysmal vascular disease.

### DESCRIPTION OF THE BACKGROUND ART

Angioplasty has become a generic term, which refers to a myriad of ideas for opening occluded or stenotic vessels. Percutaneous transluminal coronary angioplasty (PTCA) and percutaneous transluminal angioplasty (PTA) procedures for treating a patient having a stenotic (constriction), or occluded (closed) blood vessel in a coronary or peripheral artery, have become widely accepted therapeutic alternatives to coronary and peripheral arterial bypass surgery for many patients. PTCA and PTA increase the vessel lumen by radial expansion of the plaque or other pathology through “controlled” tearing of the vessel lining. The principal advantage of the PTCA or PTA procedure over other surgical procedures is its ability to enlarge the narrowed vessel or recanalize the occluded vessel at a lower or reduced morbidity and mortality than its surgical alternative, as well as, eliminating the immediate surgical postoperative discomforts, reducing hospital costs, and more rapidly returning the patient to work or allowing performance of activities of daily life. These constructs and concepts, often under the term of endovascular or endoluminal surgery, can now be applied to aneurysmal disease by reconstructing a vessel using endoluminal graft prostheses which permits recreation of the blood flow lumen and tensile strength reinforcement of the aneurysm and the cavity outside the blood lumen so as to prevent aneurysmal rupture.

Over the last several years, the introduction of endoluminal graft prostheses, such as stents, using endovascular or endoluminal surgical techniques for treatment of arterial and venous defects, such as aneurysms, have provided promise of a technique whose procedural morbidity and mortality may be significantly lower than that of surgical alternatives. Experimental studies using stents, with or without endovascular coil implantation in surgically created canine or porcine abdominal aortic aneurysms, have demonstrated successful aneurysmal exclusion. One study comparing the use of covered and uncovered (bare metal) self-expanding stainless steel stents with and without vascular coil embolization revealed that an aneurysmal cavity was excluded from arterial circulation in animals with covered as well as uncovered stents. The animals receiving bare metal stents had completely clotted aneurysms, which had markedly decreased in size and widely, patent major arterial branches.

Histology of the bare metal stents revealed a thin layer of neointimal composed primarily of myoblastic-like cells, with little reaction in the underlying aorta. Histology of the covered stent devices, following necropsy, revealed variable endothelialization of the surface of the stent, and the coating fabric was permeated by a fibroblastic and histiocytic reaction with patchy areas of chronic inflammation. Thus, uncovered stents were able to cause a reduction in size of the created aneurysms, while providing a framework for neointimal growth without occluding the side branches. While the immediate aneurysm size reduction was less pronounced in the uncovered stent cohort, the bare metal stent cohort, with or without vascular coil embolization, significantly reduced the size of, or resulted in complete thrombosis of the aneurysms at 4-week follow-up.

These data may be interpreted as the mechanism of the thrombosis with bare metal stents was related to induction of shear forces introduced by the wires across the mouth or opening of the aneurysm which reduced laminar flow creating turbulence causing thrombosis. This redirection of flow, away from the dilated aortic wall, allowed for a reduction in the wall tension, and contraction of the aneurysm.

However, even with rapid advances in stent graft technology, technologic dilemmas remain which influence procedural success, procedurally related complications, and applicable patient populations. These problems are often related to the stents themselves, because of their large profile, rigid design, method of expansion, radial force and hoop strength, and difficulty in creating fluid tight seals proximally, and distally. Present devices have resulted in (limb) vessel thrombosis, distal thromboembolism, endoleak (acutely, or during follow-up), side branch occlusion, and single limb occlusion in a bifurcated system. As a result, stent graft technology procedures typically require general or regional anesthesia, and surgical exposure for vascular access and/or repair which create additional risks for the patient.

Furthermore, the human vascular tree is far from uniform in structure and each procedure is a unique experience requiring the availability of a larger cadre of devices during the repair procedure. An aneurysm existing in a straight vessel segment can be excluded with a tubular graft, which also allows more simple reinforced clot creation within the aneurysm cavity. Endovascular aneurysm repair procedures are more complex when the aneurysm occurs at, abuts, or includes the bifurcation and/or extends from a region where a side branch exists. When anatomy demands a custom system to accomplish the vascular repair so as to overcome a length or diameter sizing problem, then another source of future problem exists, endoleaks occurring at the modular juncture point, or even immediate or subsequent separation of the parts, or kinking.

Repairing an aneurysm adjacent to a bifurcated vessel presents technical difficulties which include an inability to easily enter both vessel branches because of vessel size, vessel tortuosity, device size, or flexibility, and an inability to adequately expand the device and create fluid seals at the ends of the aneurysm. Moreover, if the custom device does not fit, surgical intervention may be necessary to remove the device exposing the patient to additional risk.

### SUMMARY OF THE INVENTION

The present invention provides a laminated prosthesis formed from tubular layers individually deployed in vivo using endovascular techniques and material. The tubular layers define a lumen through a diseased portion of a vascular system that is constructed in vivo. Each layer may be constructed using overlapping tubular members to provide a custom prosthesis. The general objective of providing a prosthesis for repairing a vascular defect using endovascular techniques is accomplished by constructing the prosthesis from expandable or self-expanding tubular members that are assembled in vivo.

An objective of the present invention is to provide a prosthesis for repairing complex vascular structures, such as bifurcated vessels. This is accomplished by constructing the prosthesis having at least one tubular layer composed of overlapping tubular members that are deployed to conform with the vascular structure surrounding the diseased area.

Another objective of the present invention is to provide a prosthesis that decreases the pressure in an aneurysm cavity. This is accomplished by providing a plurality of semipermeable tubular members forming a multilayer, laminated structure which attenuates pressure inside the surrounding aneurysm and allows the formation of a clot in the surrounding aneurysm cavity.

The foregoing and other objects and advantages of the invention will appear from the following description. In the description, reference is made to the accompanying drawings which form a part hereof, and in which there is shown by way of illustration a preferred embodiment of the invention.

### BRIEF DESCRIPTION OF THE DRAWINGS

FIG. 1 is a cross section view of a prosthesis incorporating the present invention;

FIG. 2 is an expanded cut away perspective view of the prosthesis of FIG. 1;

FIG. 3 is a detailed cross section view along line 3-3 of FIG. 2;

FIG. 4 is the same as FIG. 3 with clot material inserted into a cavity formed by the aneurysm;

FIG. 5 is a cross section view of a partially deployed tubular member of the present invention; and

FIG. 6 is a cross section view of a prosthesis of the present invention deployed in a bifurcated vessel.

### DETAILED DESCRIPTION OF THE PREFERRED EMBODIMENTS

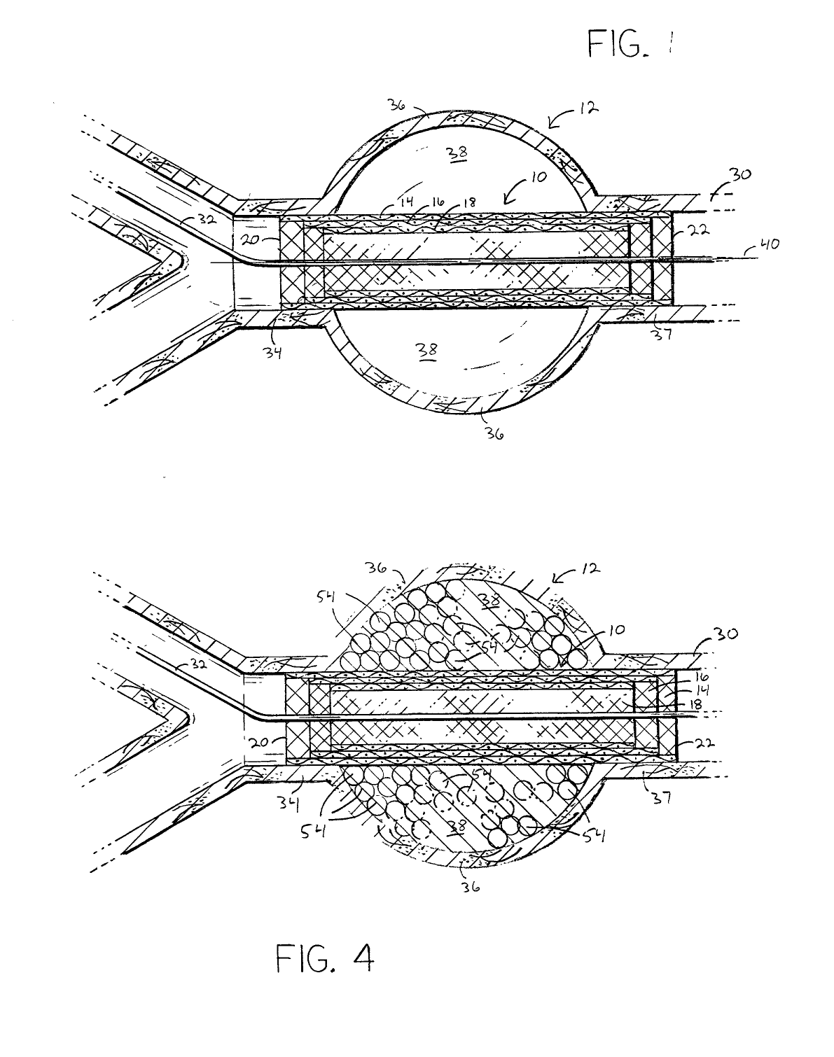

Referring to FIGS. 1 and 2, a prosthesis 10 for repairing a vascular defect 12 has an outer layer 14, an intermediate layer 16, and an inner layer 18 forming a laminated structure through the vascular defect 12. The prosthesis 10 may be assembled by individually deploying each layer 14, 16, and 18 or part thereof in vivo. Advantageously, assembling the prosthesis 10 in vivo allows the creation of a custom prosthesis that can be percutaneously deployed to repair the vascular defect 12 without the disadvantages of large preassembled alternatives.

As shown in FIG. 1, the vascular defect 12 may, for example, be a localized pathological, blood filled dilation of a blood vessel 30 caused by a disease or weakening of a blood vessel wall 36 forming an aneurysmal cavity 38. Advantageously, the prosthesis 10 of the present invention maintains the blood lumen size defined by the non-diseased portions of the blood vessel 30 and allows the aneurysmal cavity 38 external to the new lumen formed by the prosthesis 10 to become filled with stronger blood clot (not shown). The blood clot attenuates the blood pressure on the vascular defect 12, thus reducing the risk of an aneurysm rupture. Although an aneurysm vascular defect 12 is described herein, the vascular defect may also be an obstruction, stenosis, dissection, clot, weakened vessel wall or the like without departing from the scope of the present invention.

Each layer 14, 16, and 18 is substantially tubular forming a lumen through the vascular defect 12 and may be formed from materials, such as synthetic polymers, bipolymers, genetically modified endothelial cells, and other materials known in the art and as described herein. Each layer material is selected to impart desirable properties or functions to the prosthesis, such as structural rigidity, porosity, drug delivery, branching of collaterals, or the like. Although three layers are described herein, the prosthesis 10 may be formed from one or more layers without departing from the scope of the present invention.

As shown in FIG. 1, the outer layer 14 is disposed within the blood vessel 30, orientated substantially parallel to the longitudinal axis 40 of the blood vessel 30, and engages the blood vessel 30 at a proximal end 20 and a distal end 22. The outer layer proximal end 20 engages a normal (non-diseased) or at least only a relatively unaffected (nominally diseased) arterial vessel 34 upstream to the aneurysm 12. The prosthesis outer layer distal end 22 engages a normal or relatively unaffected vessel segment 37 located downstream to the aneurysm 12 forming a lumen through the vascular defect 12 from the proximal end 20 to the distal end 22. The outer layer may also have a portion at each end 20, 22 defining a longitudinally-oriented slot (not shown) enabling tissue to grow therethrough to anchor the prosthesis within the vessel.

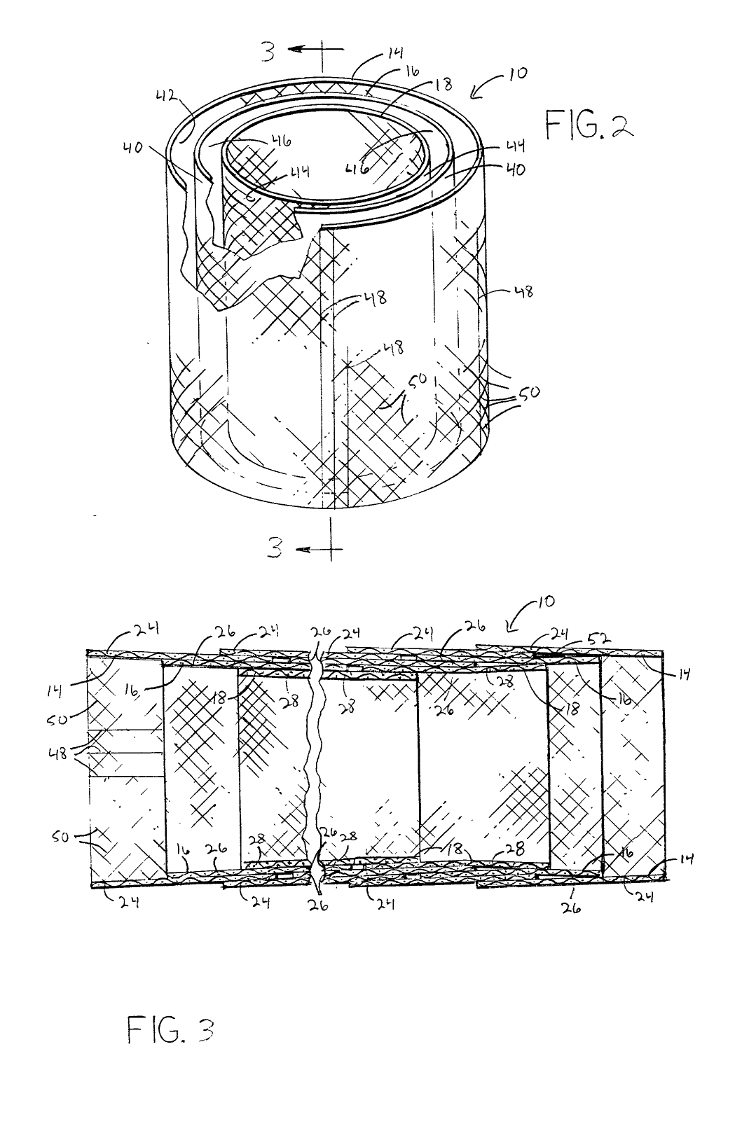

Referring particularly to FIG. 3, the prosthesis outer layer 14 is preferably constructed in vivo by sequentially introducing one or more thin walled tubular members 24 in an overlapping relationship. Preferably, the outer layer 14 has a length greater than any single tubular member 24 to allow greater flexibility in customizing the prosthesis 10 for the particular vascular defect 12 or vessel configuration.

Each tubular member 24 is expandable substantially uniformly over its entire length from a relaxed, small diameter to one or more larger diameters to define the lumen. The tubular members 24 may be expanded by a balloon, self-, or thermal expansion, or some other similar releasing mechanism system known in the art. Preferably, the tubular members 24 are self expanding to avoid complicating the deployment procedure. The radial force of the expanded tubular members and the intrinsic hoop strength of the layers 14, 16, and 18 hold the members 24 together.

Individual outer layer tubular members 24 are deployed in vivo in an overlapping relationship to customize the prosthesis 10 for various lengths, shapes, and strength requirements. Advantageously, the individual tubular members 24 are of small caliber and can be easily introduced through significantly smaller diameter catheter(s) and sheaths than fully assembled or modular prostheses, obviating the need for general anesthesia or vascular surgical exposure for arterial repair.

Preferably, each outer layer tubular member 24 is composed of a semi-permeable or impermeable material, such as a nitinol, stainless steel, or polymeric mesh, to provide a structural framework for the prosthesis 10 and sufficient flexibility and porosity to allow the placement of material within the aneurysmal cavity 38, external to the cylindrically shaped prosthesis 10. In a preferred embodiment, more clearly shown in FIGS. 2 and 3, the tubular members 24 are a mesh having a plurality of longitudinal members 48 interconnected by serpentine members 50 inclined at an angle with respect to the longitudinal members, such as described in U.S. Pat. Nos. 5,314,444 and 5,758,562, which are incorporated herein by reference.

Preferably, at least one of the tubular members 24 is covered by a biocompatible material to encourage neointinal growth or incorporates bioactive materials for in vivo release. The bioactive materials, such as synthetic fiber covered coils, cyanoacrylates, polymers, stainless steel coils, clotting agents, biocompatible polymeric materials, genetically modified endothelial cells, or the like, may be released either into the tissue, to the lumen surface, or the interior of the lumen providing distinct advantages inherent to the released material. For example, a bioactive material, such as a clotting agent, released into the aneurysmal cavity increases the tensile strength of the clot external to the prosthesis in conjunction with the fibrin meshwork of the prosthesis 10.

Preferably, the outer layer tubular members 24 have specific properties, such as a fixed maximum diameter which provides for a maximum lumen size. Other desirable properties, such as low profile, flexibility, porosity, structural framework allow for placement of devices to deliver bioactive materials to the outer layer 14 or external to the outer layer 14 to form an external clot which has an increased tensile strength. The tubular members 24 having specific properties are selected depending upon the specific requirements to repair the vascular defect 12.

An adhesive 52, such as a collagen based adhesive or cyanoacrylate, may be added which joins and holds the tubular members 24 together. An adhesive or thrombus itself enables fibrin to be insinuated between and among porous interstices of the overlapping portions of the tubular members 24 binding them together. The adhesive 52 may also be employed to anchor the outer layer 14 to the normal or relatively unaffected vessel segments, 34, 37.

As shown in FIG. 4, once the outer layer 14 has defined a tubular lumen, a clot inducing material 54 for increasing the thrombus tensile strength is introduced into the aneurysm cavity 38. This clot inducing material 54 may be thrombogenic or vasoocclusive coil(s), such as available from Cook Incorporated, Bloomington, Indiana, Alternatively, the clot inducing material 54 may be a fluffy material composed of fibrils, or a biocompatible polymeric material, which has a fluent state and allows application, delivery, and upon contact with blood, an increased or altered less fluent or non-fluent state in vivo. The clot inducing material 54 is introduced into the aneurysm cavity 38 using methods known in the art, such as through small, plastic catheters, hollow guide wires, needles, or the like.

Referring back to FIG. 2, the intermediate layer 16 is disposed within the lumen defined by the outer layer 14 and has an outer surface 40 engaging an inner surface 42 of the outer layer 14 providing additional structural integrity to the prosthesis 10. As in the outer layer 14, preferably, the intermediate layer 16 is composed of a plurality of expandable, overlapping semi-permeable or impermeable tubular members 26.

As shown in FIG. 3, the intermediate tubular members 26 forming the intermediate layer 16 are preferably deployed within the central lumen formed by the outer layer 14 from the outer layer proximal end 20 and then the distal end 22 so as to overlap in the central lumen providing increased structural stability. Intermediate layer tubular members 26 may be composed of a material such as described for the outer layer 14. For example, nitinol, stainless steel, or a polymeric mesh may be used to provide added strength to the prosthesis. Additionally, one or more intermediate layer tubular members may incorporate biocompatible material for release once deployed.

Preferably the intermediate layer 16 is composed of semipermeable tubular members 26 to provide attenuation of pressure inside the aneurysm cavity 38 and allow the formation of a clot reducing the pressure in the cavity 38. As in the outer layer 14, an adhesive 52 may be added which joins and holds the tubular members 26 together. The adhesive 52 may also be employed to bind the intermediate layer 16 to the outer layer 14, or the inner layer 18.

Referring back to FIG. 2, the inner layer 18 is disposed inside the intermediate layer 16 and has an outer surface 44 engaging an inner surface 46 of the intermediate layer 16. An expandable inner layer 18 maintains expansion of the prosthesis 10 and provides support to the laminated structure. Advantageously, a self expanding inner layer 18 attenuates the blood pressure in the aneurysm cavity 38.

As shown in FIG. 3, the inner layer 18 may be composed of tubular members 28 as described for the outer layer 14 and intermediate layer 16. Preferably, the inner layer 18 comprises overlapping, expandable, tightly woven, knitted or braided thin tubular members 28, such as a polymeric mesh, stainless steel, nitinol or other alloys, to form a smooth lining within the lumen created by the intermediate layer 16 and prevent post intervention complications.

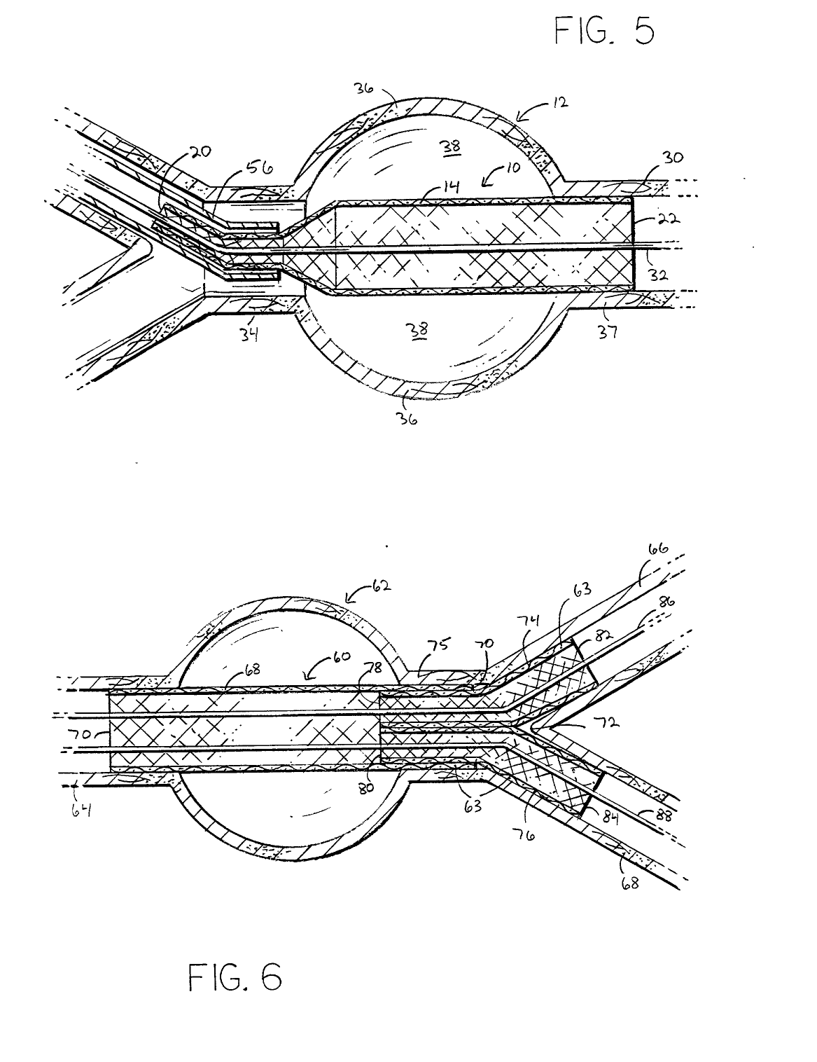

As illustrated in FIGS. 5 each layer 14, 16, 18 of the prosthesis 10 is deployed into the diseased area of the vascular defect 12 using an interventional procedure initiated by obtaining vascular access through a percutaneous approach or small surgical incision. Advantageously, percutaneous access and local anesthesia provides the opportunity to apply materials, such as synthetic polymers, bipolymers, clotting agents, genetically modified endothelial cells and the like, to the prosthesis layers 14, 16, 18 or external to the layers 14, 16, 18 directly into the aneurysm cavity 38.

A delivery system, such as a guide wire 32 introduced into the vessel 30 through a hemostatic vascular sheath 56 for guiding a catheter (not shown) to the vascular defect 12, may be used to deploy the prosthesis 10. This is usually followed by a bolus of heparin, which is administered intravenously to achieve adequate anticoagulant effect and to prevent vascular thrombosis.

The catheter is then advanced to the site of the vascular defect 12 through the hemostatic sheath 56 and over the guide wire 32. The catheter transports the prosthesis tubular members 24, 26, and 28 to the diseased area forming the prosthesis 10 in vivo.

Each prosthesis tubular member, 24, 26, and 28 is deployed using balloon, self-, or thermal expansion, or some other similar releasing mechanism system known in the art. The assembled prosthesis 10 expands outward from a contracted configuration to a fully deployed configuration to recreate a tubular lumen infrastructure within the vascular defect 12, and does not come in contact with the aneurysm wall 36 except at the ends of the vascular defect 12 where the lumen is the size of a normal or intact vessel 30. Although, the prosthesis 10, as described above, is expanded into place after the layers 14, 16, and 18 have been assembled in their final positions, each tubular member 24, 26, and 28 can be inserted and expanded individually to form the completed prosthesis 10 without departing from the scope of the present invention.

In a second embodiment, shown in FIG. 6, a prosthesis 60 for repairing a diseased segment, such as an aneurysm 62, of a bifurcated or otherwise non-uniform vessel in a vascular system has at least one layer 63 formed from a plurality of tubular members 68, 74, and 76 as described for the first embodiment. A bifurcated artery 75 has a main blood vessel 64 branching into a first branch 66 and a second branch 68. The aneurysm 62 in the main blood vessel 64 and adjacent to the branches 66, 68 requires a custom prosthesis 60 to avoid occluding one of the branches 66, 68.

The intraluminal prosthesis 60 traverses the fluid containing aneurysm 62 without branch occlusion. As shown in FIGS. 6, the main tubular member 68 having a proximal end 70 engaging a normal or relatively unaffected portion of the main blood vessel 64 and distal end 70 terminating proximal to the bifurcation point 72 of the main blood vessel 64. Two branch tubular members 74, 76 have a proximal end 78, 80 disposed in the distal end 70 of the main tubular member 68 in an overlapping relationship and a distal end 82, 84 extending into one branch 66, 68 of the bifurcated vessel. The distal end 82, 84 of each branch tubular member 74, 76 engages a normal or relatively unaffected portion of the respective branches 66, 68 forming a bifurcated outer layer.

Subsequent layers are then deployed as described above for the first embodiment reinforcing the structural integrity of the prosthesis 60 and preventing leaks at the joint between the branch outer layers 74, 76 extending into each branch 66, 68 of the bifurcated vessel and the main outer layer 68. Each layer may be formed from one or more tubular members as described for the first embodiment.

Alternatively, a bifurcated outer layer may be formed by passing a smaller tubular member through a slit or window formed in a side of a tubular outer member. Subsequent layers are then deployed as described above to provide the laminated structure of the present invention.

Deployment of the prosthesis 60 for repairing a diseased segment of a bifurcated vessel follows the same procedure as disclosed above. However, deploying tubular members of the present invention in individual vessel branches may require a delivery system which includes a bifurcated endovascular catheter as described in U.S. Pat. No. 5,720,735, which is fully incorporated herein by reference. A bifurcated endovascular catheter allows simultaneous deployment of two tubular members or a single bifurcated tubular member over separate guide wires 86, 88 preventing occlusion or collapse of one of the branches.

The deployment and construction methodology disclosed herein enables placement of the prosthesis to an exact bifurcation level, as well as, origin of the renal arteries. Advantageously, the methodology precludes migration or embolization of the extraluminally placed coils or increasing tensile strength material and precludes invagination of the device upon itself as the aneurysm shrinks and the length of blood flow lumen shortens with de-rotation of the aneurysm. This lamination technique also enables aneurysm exclusion and limb creation without the need for watertight seals of the limbs in the main aortic shaft.

While there has been shown and described what are at present considered the preferred embodiment of the invention, it will be obvious to those skilled in the art that various changes and modifications can be made therein without departing from the scope of the invention. For example, the present invention as described herein is used to repair an aneurysm, the present invention may also be used to direct fluid flow through a lumen in an organ. Therefore, any references to a vessel, also includes an organ.

We claim:

1. A laminated prosthesis for repairing a diseased area in a vessel comprising:
a first tubular layer having a proximal end and distal end defining a lumen through said diseased area, said proximal end engaging a first surface of said vessel upstream of said diseased area and said distal end engaging a second surface of said vessel downstream of said diseased area; and
a second tubular layer disposed in said first tubular layer, said second tubular layer being deployed in said first tubular layer in vivo.

2. A laminated prosthesis as in claim 1, wherein said first surface of said vessel is nominally diseased.

3. A laminated prosthesis as in claim 1, wherein said second surface of said vessel is nominally diseased.

4. A laminated prosthesis as in claim 1, wherein one of said layers is formed from one or more tubular members,

5. A laminated prosthesis as in claim 4, wherein one or more of said tubular members is deployed in vivo.

6. A laminated prosthesis as in claim 4, wherein at least one of said tubular members are shorter than said layer formed therefrom.

7. A laminated prosthesis as in claim 1, wherein at least one of said layers is a mesh scaffolding.

8. A laminated prosthesis as in claim 7, wherein said mesh comprises a plurality of longitudinal members interconnected by serpentine transverse members.

9. A laminated prosthesis as in claim 1, wherein at least one of said layers includes a material selected from the group consisting of nitinol, stainless steel, synthetic polymers, bipolymers, genetically modified endothelial cells, biocompatible materials, permeable materials, semipermeable materials, and impermeable materials.

10. A laminated prosthesis as in claim 1, wherein at least one of said layers expands from a contracted configuration to a fully deployed configuration.

11. A laminated prosthesis as in claim 1, wherein at least one of said layers has a portion defining a longitudinally-oriented slot, the longitudinally-oriented slot enabling tissue to grow therethrough to anchor the prosthesis within the vessel.

12. A laminated prosthesis as in claim 1, wherein said first layer defines a bifurcated lumen.

13. A laminated prosthesis as in claim 1 wherein said tubular members are bonded together by an adhesive.

14. A prosthesis for repairing a diseased area in a vessel comprising:
a first tubular member having a proximal end and a distal end, said proximal end engaging a first surface of said vessel outside of said diseased area and said distal end extending into said diseased area; and
one or more overlapping other tubular members, each other tubular member having a proximal end and a distal end, wherein a proximal end of one of said other tubular members being in an overlapping relationship with said first tubular member and a distal end of one of said other tubular members engaging a second surface of said vessel outside of said diseased area, wherein said first and other tubular members define a lumen through said diseased area.

15. A prosthesis as in claim 14, wherein one or more of said tubular members are individually deployed in vivo.

16. A prosthesis as in claim 14, wherein at least one of said layers is a mesh scaffolding.

17. A prosthesis as in claim 16, wherein said mesh scaffolding comprises a plurality of longitudinal members interconnected by serpentine transverse members.

18. A prosthesis as in claim 14, wherein said first surface of said vessel is nominally diseased.

19. A prosthesis as in claim 14, wherein said second surface of said vessel is nominally diseased.

20. A prosthesis as in claim 14, wherein at least one of said tubular members includes a material selected from the group consisting of nitinol, stainless steel, synthetic polymers, bipolymers, genetically modified endothelial cells, biocompatible materials, permeable materials, semipermeable materials, and impermeable materials.

21. A prosthesis as in claim 14, wherein at least one of said tubular members expands from a contracted configuration to a fully deployed configuration.

22. A prosthesis as in claim 14, wherein at least one of said tubular members has a portion defining a longitudinally-oriented slot, the longitudinally-oriented slot enabling tissue to grow therethrough to anchor the prosthesis within the vessel.

23. A prosthesis as in claim 14, wherein at least two of said tubular members are bonded together by an adhesive.

24. A prosthesis as in claim 14, wherein said tubular members define a bifurcated lumen.

25. A method of directing flow through a blood vessel, the method comprising steps of:
disposing a first tubular member in a contracted configuration on a delivery system;
advancing the delivery system inside said vessel to dispose the first tubular member at the desired location within the vessel;
actuating the delivery system to deploy the first tubular member at the desired location;
disposing a second tubular member in a contracted configuration on a delivery system;
advancing the delivery system along a guide wire to dispose the second tubular member at the desired location within the vessel; and
actuating the delivery system to deploy the second tubular member in an overlapping relationship with said first tubular member forming a prosthesis having an external wall.

26. The method as defined in claim 25 further comprising the step of progressively adding additional tubular member in an overlapping relationship, said tubular members forming layers of materials having different properties.

27. The method as defined in claim 25 further comprising the step of introducing an adhesive to bond said tubular members together.

28. The method as defined in claim 25 further comprising the step of introducing material inside a cavity between the external wall of the prosthesis and an internal wall of the vessel.

29. The method as defined in claim 28, wherein said material is selected from the group consisting of coils, fibers, glues, hydrocarbons, gels, cyanoacrylates.

30. The method as defined in claim 28, wherein said material is introduced inside said cavity using a catheter.
